SEQUENCE LISTING (3 sequences)

SEQ ID NO: 1 - protein, 29 aa, Canis familiaris
  MOD_RES (1)..(1): ACETYLATION
NPGFPPLVAPEPDALCAAFQDNEQLFLGK

SEQ ID NO: 2 - protein, 29 aa, Felis catus
  MOD_RES (1)..(1): ACETYLATION
NPGFGQLVTPEADAMCTAFHENEQRFLGK

SEQ ID NO: 3 - protein, 29 aa, Artificial Sequence
  consensus sequence between SEQ ID NOS 1 and 2
NPGFXXLVXPEXDAXCXXFXXNEQXFLGK

(19) United States
(12) Patent Application Publication
(10) Pub. No.: US 2013/0266972 A1
(43) Pub. Date: Oct. 10, 2013

(54) METHOD OF DETECTING PROTEIN LOSING ENTEROPATHY IN ANIMALS

(71) Applicant: Seth FISHMAN, Highland Beach, FL (US)

(72) Inventor: Seth FISHMAN, Highland Beach, FL (US)

(21) Appl. No.: 13/837,023

(22) Filed: Mar. 15, 2013

Related U.S. Application Data
(63) Continuation-in-part of application No. 12/283,653, filed on Sep. 15, 2008, now abandoned.
(60) Provisional application No. 60/993,651, filed on Sep. 13, 2007.

(51) Int. Cl. G01N 33/68; C07K 16/18; C07K 14/765

(52) U.S. Cl. CPC G01N 33/6893; C07K 14/765; C07K 16/18
USPC 435/7.92; 436/501; 530/324; 530/387.9

(57) ABSTRACT

The present invention provides a method and kit for the detection of Protein Losing Enteropathy (PLE) in animals. The method includes the steps of (a) obtaining a biological sample from an animal to be tested and (b) determining the absence or presence of albumin in the biological sample via a kit comprising an immunoassay utilizing a species-specific anti-albumin antibody.

### CROSS-REFERENCE TO RELATED APPLICATIONS

This is a continuation-in-part of application Ser. No. 12/283,653, filed Sep. 15, 2008, pending; which claimed priority benefit of Application No. 60/993,651, filed on Sep. 13, 2007; the contents of which are incorporated herein by reference in their entirety.

### BACKGROUND OF INVENTION

Protein losing enteropathy refers to any condition of the gastrointestinal tract in humans and animals that results in a net loss of protein from the body. Common causes of protein losing enteropathy include celiac disease, Crohn's disease, short bowel syndrome (where the absorptive area for proteins is decreased), intestinal lymphangiectasia, amyloidosis, enteropathy caused by NSAIDs, and giardiasis. The diagnosis of protein losing enteropathy is typically made by excluding other causes of protein loss, such as nephrotic syndrome. Endoscopy and barium imaging can be used to localize the cause of the protein loss in the bowel. However, these methods are costly, time consuming, and generally only feasible in humans. A need therefore exists for a relatively inexpensive and quick method and kit for the detection of protein losing enteropathy in animals.

### SUMMARY OF INVENTION

The present invention relates to a method and kit for the detection of Protein Losing Enteropathy (PLE) in animals. Preferred animals to test for PLE are domesticated animals with horses, cows, dogs, cats, and small reptiles being the most preferred. Method and kit embodiments disclosed herein are based on discovering the absence or presence of albumin in a biological sample from an animal as an indicator of PLE. The most preferred method or kit embodiment is an immunoassay utilizing a species-specific anti-albumin antibody.

### DETAILED DESCRIPTION OF SPECIFIC EMBODIMENTS

The present invention relates to a method and kit for the detection of Protein Losing Enteropathy (PLE) in animals. PLE is the loss of blood serum proteins into fecal matter as a result of compromised epithelial tight junctions in the gastrointestinal tract. Under normal circumstances, tight junctions between epithelial enterocytes in the gastrointestinal tract by virtue of their tight junctions prevent the leakage and or loss of various molecules and proteins from the intravascular space into the gastrointestinal lumen. Various conditions ranging from inflammatory, infectious (bacterial, viral and parasitic), to autoimmune may cause inflammation and or edema, which may then disrupt the tight junctions, resulting in hypoalbuminemia, hypoproteinemia, and severe to possibly fatal fluid retention in various body compartments such as the chest. A major indicator of PLE is the presence or increased concentration of serum albumin in the fecal mater.

A method of the present invention can be generally accomplished by:
(a) obtaining a biological sample from an animal; and
(b) determining the absence or presence of albumin in the biological sample via a kit comprising an immunoassay utilizing a species-specific anti-albumin antibody.

As used herein, the term “biological sample” is meant to encompass any specimen obtained from an animal that can be used to measure albumin leakage through the epithelial tight junctions of the gastrointestinal tract, with fecal matter being the most preferred.

As used herein, the term “animal” is meant to encompass any non-human organism capable of developing PLE. Preferred animals to test for PLE are domesticated animals with horses, cows, dogs, cats, and small reptiles being the most preferred.

As used herein, the term “kit” is meant to encompass any device capable of being contacted by a biological sample and comprising an immunoassay utilizing a species-specific albumin antibody.

As used herein, the term “assay” is meant to encompass any immunoassay designed to use antibodies including, but not limited to: sandwich immunoassay, radioimmunoassay, enzyme-linked immunosorbant assay (ELISA), sandwich enzyme-linked immunosorbant assay, fluorescence immunoassay, competitive exclusion immunoassay, radial diffusion immunoassay, “dipstick” immunoassay, and laminar flow immunoassay. The preferred assay is an immunoassay designed to use antibodies antigenic to the serum albumin of the desired species to detect the presence of serum albumin in a biological sample, most preferably in fecal matter.

As used herein, the term “antibody” is meant to encompass any antibody antigenic to serum album. The antibodies used in the present invention can be polyclonal, monoclonal, or antibody fragments (FAB). The antibodies for the present invention may be prepared in either the classic methods of animal inoculation and isolation from blood serum, formation of cell line hybridomas and establishment of cell cultures producing monoclonal antibodies, or via newly developed recombinant techniques for the rapid screening, isolation, and production of monoclonal antibodies in bacterial cultures. A preferred antibody used in the present invention will be specific for the target species of the diagnostic tool (i.e. equine anti-albumin antibodies will be used in the Equine PLE Diagnostic Indicator Test; bovine anti-albumin antibodies will be used in the Bovine PLE Diagnostic Indicator Test; canine anti-albumin antibodies will be used in the Canine PLE Diagnostic Indicator Test; feline anti-albumin antibodies will be used in the Feline PLE Diagnostic Indicator Test, reptilian anti-albumin antibodies will be used in the Reptilian PLE Diagnostic Indicator Test, etc.).

In one embodiment, the kit is an analytical test device incorporating a porous solid phase material carrying in a first zone a labeled reagent that is retained in the first zone while the porous material is in the dry state but free to migrate through the porous material when the porous material is moistened by the application of the biological sample to the sample pad; the porous material carrying in a second zone, which is spatially distinct from the first zone, an unlabelled specific binding reagent having specificity for albumin and is capable of participating with the labeled reagent in either a “sandwich” or a “competition” reaction, the unlabelled specific binding reagent being firmly immobilized on the porous material such that it is not free to migrate when the porous material is in the moist state. The kit will include instructions on the method of: (a) obtaining a biological sample from an animal to be tested and (b) determining the absence or presence of albumin in the biological sample via the included immunoassay.

In one embodiment, the porous solid phase material is a nitrocellulose membrane.

In one embodiment, the labeled reagent is a species-specific anti-albumin antibody.

In one embodiment, the assay will be a laminar flow immunoassay (LFIA) that utilizes antibodies specific for albumin to detect the presence or absence of such in the biological sample.

In one embodiment, the assay will be considered a “sandwich” assay as described by David et al. (U.S. Pat. No. 4,376,110), herein incorporated in its entirety. The preferred form of this assay has been generally described by May et al. (U.S. Pat. No. 5,602,040), herein incorporated in its entirety.

In one embodiment, the present invention will use species-specific anti-albumin antibodies immobilized on a nitrocellulose membrane as the capture antibody. Additionally, a control line of immobilized antibody specific for other antibodies will be painted on the nitrocellulose membrane after the test line of anti-serum albumin antibodies. A mobile phase, labeled (fluorescent or colormetrically labeled) anti-albumin antibody will be contained in the sample pad. When the biological sample is applied to the sample pad, the sample will interact with the mobile phase, labeled anti-albumin, and will elute across the nitrocellulose membrane. If there is albumin analyte present in the biological sample, the mobile phase, labeled antibodies will bind with the albumin present in the sample, thus attaching a tag to the analyte. The tagged analyte will migrate across the nitrocellulose membrane where it will additionally interact with and bind to the immobilized anti-albumin antibodies, thus forming a line. If no albumin analyte is present in the sample, the tagged anti-serum albumin antibodies in the mobile phase will migrate, unattached to any analyte, across the nitrocellulose membrane, bypassing the test line. In all cases the final control line is specific for the tagged antibodies and will always capture these antibodies, resulting in a control line indicating the test worked as required.

### EXAMPLES

Polyclonal antibodies were produced in rabbits. Rabbits were immunized with a unique peptide sequence derived from the serum albumin protein of Canis lupus familiaris (amino acid residues from 133 to 161 of NCBI Reference Sequence: NP_{—}001003026.1; SEQ ID NO: 1 modified at N- and C-termini as described below):

Ac-NPGFPPLVAPEPDALCAAFQDNEQLFLGK-amide.

This peptide was conjugated to a non-vertebrate carrier protein and used to immunize rabbits by a standard immunization/antibody production protocol. Serum was harvested and specific antibodies isolated by affinity purification using the immunizing peptide coupled to agarose as capture reagent. Serum and affinity-purified antibodies were tested for specific reactivity to Canine Serum Albumin (CSA) by ELISA. The serum and affinity-purified antibody were both found to have high titers against the immunizing peptide, confirming the antibody production. The affinity-purified antibody generated against CSA was found be specific for CSA and was able to detect CSA at a sensitivity level of less than 5 ng/sample assay in the ELISA. In addition, when the affinity-purified antibody was tested for cross reactivity against serum albumin proteins from rabbit, goat, mouse, rat, duck, pig, cow, sheep, horse, human, and human gamma globulin, little or no reactivity was found as determined by ELISA and western blotting.

Similarly, polyclonal antibodies may be produced in rabbits by immunization with a unique peptide sequence derived from the serum albumin protein of Felis catus. The known amino acid sequence of serum albumins from different animal species can be aligned to identify a unique peptide sequence capable of generating a species-specific antibody for Feline Serum Albumin (FSA). An immunizing peptide may be selected based on sequence uniqueness and predicted immunogenicity. Serum and affinity-purified antibody may be produced as in the preceding example. The affinity-purified antibody would be tested for cross reactivity against serum albumin proteins from rabbit, goat, mouse, rat, duck, pig, cow, sheep, horse, human, and human gamma globulin to confirm little or no reactivity is found. One possible peptide sequence (amino acid residues from 133 to 161 of NCBI Reference Sequence: NP_{—}001009961.1; SEQ ID NO: 2 modified at N- and C-termini as described below) is the following:

Ac-NPGFGQLVTPEADAMCTAFHENEQRFLGK-amide

Aligning the peptide sequences for canine (SEQ ID NO: 1) and feline (SEQ ID NO: 2) serum albumins highlights differences between the two companion animals and other species, which amounts to ten amino acid residues out of a total of 29 (shown as “X” above positions different from other species; SEQ ID NO: 3):

NPGFXXLVXPEXDAXCXXFXXNEQXFLGK
Ac-NPGFPPLVAPEPDALCAAFQDNEQLFLGK-amide
Ac-NPGFGQLVTPEADAMCTAFHENEQRFLGK-amide

Based on these ten differences between the dog and cat serum albumins and other species in the primary structure shown above, species-specific antibodies can be generated that do not cross react with serum albumins ingested by the dog or cat. For example, aligning the 29 amino acid residues of SEQ ID NO: 1 with the comparable 28 amino acid residues of bovine serum albumin (BSA) protein shows 13/28 differences (gap of one residue is required to optimize alignment) in their amino acid sequences. Such antibodies generated against CSA and FSA permit the specific detection of autologous serum albumin (e.g., dog or cat) and lack of detection of serum albumin from other species, thereby distinguishing between autologous and dietary serum albumins.

In particular, it is preferred that a species-specific antibody recognize serum albumin of the animal being diagnosed (e.g., serum albumin of dog or cat) and not cross react with serum albumins found in the diet of that animal (e.g., serum albumins of chicken, cow, horse, pig, and sheep). Such a species-specific antibody permits detection of protein loss from the dog's or cat's body by distinguishing the host's serum albumin from dietary serum albumins. The immunizing peptide may be a fragment of the serum albumin protein having a length of 10 or more residues, 20 or more residues, about 30 residues, 40 or less residues, 50 or less residues, or any combination thereof (e.g., from 10 to 50 residues long). Monoclonal antibodies and antibody fragments may be produced using the same immunizing peptide.

1. A kit comprising an assay for detection of a key protein in a biological sample from an animal; wherein (i) the assay is an immunoassay, (ii) the key protein is serum albumin of the animal, and (iii) the immunoassay comprises species-specific antibody that binds the serum albumin.

2. The kit of claim 1, wherein the biological sample used is comprised of fecal matter.

3. The kit of claim 1, wherein the immunoassay is a sandwich immunoassay or a competitive exclusion immunoassay.

4. The kit of claim 1, wherein the immunoassay is an enzyme-linked immunosorbant assay (ELISA), a fluorescence immunoassay, or a radioimmunoassay.

5. The kit of claim 1, wherein the immunoassay is a laminar flow immunoassay.

6. The kit of claim 1, wherein the species-specific antibody does not detect equine serum albumin, bovine serum albumin, canine serum albumin, feline serum albumin, or any combination thereof.

7. The kit of claim 1, wherein the species-specific antibody detects only canine serum albumin or only feline serum albumin.

8. The kit of claim 1, wherein the species-specific antibody binds to at least an antigen comprising SEQ ID NO: 1 or 2.

9. The kit of claim 1, wherein the species-specific antibody is a polyclonal antibody, a monoclonal antibody, or a fragment thereof.

10. A method for diagnosis of Protein Losing Enteropathy (PLE) in an animal, the method comprising:
(a) obtaining a biological sample from an animal and
(b) detecting absence or presence of serum albumin of the animal in the biological sample using an immunoassay, which is comprised of species-specific antibody that binds the serum albumin;
wherein PLE is diagnosed in the animal when the serum albumin is detected as present in the biological sample.

11. The method according to claim 10, wherein the biological sample is comprised of fecal matter.

12. A species-specific peptide from 10 to 50 residues long and having an amino acid sequence, wherein the sequence is present in a serum albumin of one animal species and not present in other animal species.

13. The peptide of claim 12, wherein the peptide is conjugated to a detectable label or a solid phase material.

14. The peptide of claim 12, wherein the peptide is comprised of SEQ ID NO: 1 or 2.

15. An antigen conjugated to a heterologous carrier, wherein the antigen comprises the amino acid sequence of claim 12.

16. The antigen of claim 15, wherein the heterologous carrier is an immunogen, a detectable label, or a solid phase material.

17. The antigen of claim 15, wherein the amino acid sequence is SEQ ID NO: 1 or 2.

18. A species-specific antibody specific for a serum albumin of an animal species selected from the group consisting of dog, cat, horse, cow, and small reptiles; wherein the species-specific antibody binds the antigen of claim 15.

19. The antibody of claim 18, wherein the species-specific antibody is a polyclonal antibody, a monoclonal antibody, or a fragment thereof.

20. A kit comprising an immunoassay as set forth in claim 1 and at least one of: (i) a species-specific peptide, which is optionally conjugated to a detectable label or a solid phase material; (ii) an antigen conjugated to a detectable label or a solid phase material; (iii) a species-specific antibody, or (iv) any combination thereof.
